(19) United States
(12) Patent Application Publication
(10) Pub. No.: US 2014/0166771 A1
(43) Pub. Date: Jun. 19, 2014

(54) Passive Dispenser of Volatile Material

(71) Applicant: Reckitt & Colman (Overseas) Limited, Slough (GB)

(72) Inventors: Andrea Duddington, Hull (GB); Benjamin David Hindle, Hull (GB)

(73) Assignee: Reckitt & Colman (Overseas) Limited, Slough (GB)

(21) Appl. No.: 14/025,987

(22) Filed: Sep. 13, 2013

Related U.S. Application Data
(62) Division of application No. 12/674,243, filed on May 5, 2011.

(30) Foreign Application Priority Data
Aug. 23, 2007 (GB) 0716412.2

(51) Int. Cl. A61L 9/12

(52) U.S. Cl. CPC A61L 9/12
USPC 239/6; 239/58

(57) ABSTRACT

A non-automated dispenser adapted to passively emanate a volatile material at room temperatures via a membrane, in particularly concerned with the emanation of a volatile material such as a fragrance/air freshener, an insecticide, a disinfectant, a bactericide, a fungicide and/or a medicament.

### FIELD OF THE INVENTION

The present invention relates to a dispenser adapted to passively emanate a volatile material at room temperatures via a membrane, and particularly but not exclusively, to emanate a volatile material such as a fragrance/air freshener, an insecticide, a disinfectant, a bactericide, a fungicide and/or a medicament.

### BACKGROUND

Conventional membrane-containing emanation devices, such as devices found for the emanation of volatile liquid air fresheners, generally consist of a housing holding a quantity of the liquid air freshener therein and a membrane in constant contact on one side thereof with the liquid and exposed to the external environment on its other side. The contact between the volatile liquid and membrane causes the membrane to uptake the liquid by capillary action and/or diffusion, thus permitting the emanation of the liquid by evaporation from its surface exposed to the environment.

Whilst such devices are simple to construct they contain several drawbacks, some of which are identified below.

One drawback, particularly with the emanation of air fresheners is that of a phenomenon called “habituation”. Habituation is when users of a continuously emanating air freshener get so used to the fragrance of the air freshener that they become unable to notice it after a period of time. This is problematic in particular with non-automated air freshening devices where the user is given no visual clues as to whether the device is emanating a fragrance or not.

A further drawback associated with devices having a membrane in constant contact with a volatile material, such as a volatile liquid air freshener, is the phenomena of vacuum build-up. The vacuum build-up phenomena can occur inside the housing due to the wetted membrane being unable to allow sufficient flow of air therethrough in order to equalise the drop in pressure inside the housing caused by the emanation of the volatile material. This build-up phenomena can cause unacceptable performance problems with such a device from a consumer perspective.

In the field of air freshening it is generally preferred to use a volatile liquid fragrance/air freshener comprising several components. These components often possess different volatilities which can lead to emission problems. In devices having a membrane in constant contact with the volatile liquid, an accumulation of volatile liquid components with the lowest comparative volatilities can occur leading to an undesirable non-uniform emanation profile for the liquid.

In either or both cases of build-up phenomena and accumulation of low volatility components, the evaporation rate, and thus the emanation rate, of the volatile liquid adopts the profile shown in Fig. A.

Eventually the emanation rate may reach equilibrium (dashed line) where the rate of evaporation of each individual component of the volatile liquid away from the membrane surface is equivalent to the rate of deposition due to diffusion and the device cannot maintain any further vacuum.

Accordingly, it is an object of the present invention to provide a device that is capable of addressing the abovementioned drawbacks and other drawbacks that will be appreciated by a person skilled in the art.

### SUMMARY OF INVENTION

According to a first aspect of the present invention therefore, there is provided a passive dispenser for volatile material comprising a housing with an inner chamber configured to hold a quantity of volatile material therein, and further comprising at least two discrete membranes wherein said chamber is configured to permit direct contact between the volatile material and said membranes, and wherein, in use, the dispenser is orientated such that at least one of said membranes is not in direct contact with the volatile material.

According to a second aspect of the present invention therefore, there is provided a passive dispenser of volatile material comprising a housing with an inner chamber configured to hold a quantity of volatile material therein, the volatile material being provided in said chamber, and further comprising at least two discrete membranes wherein said chamber is configured to permit direct contact between the volatile material and said membranes, and wherein, in use, the dispenser is orientated such that at least one of said membranes is not in direct contact with the volatile material.

In the context of the present invention, the term “passive” is understood to mean the ability to emanate a volatile liquid without the assistance of electrically powered emanation means or propellant-powered aerosol systems or the like.

The membrane(s) not in contact with the volatile material may advantageously provide the dispensers according to the present invention with the ability to at least overcome the build-up phenomena associated with the prior art.

Furthermore, and as will be discussed below, the additional drawbacks of habituation and avoiding the accumulation of low volatility components may also be advantageously addressed by the dispensers according to the present invention.

The housing of the dispenser preferably comprises a front wall and a rear wall spaced apart by side edges, or a side edge where the housing is of a generally circular or elliptical shape when viewed from a side, thus defining the inner chamber. In one preferred embodiment the housing is provided in a generally circular or elliptical shape when viewed from a side. In an alternatively preferred embodiment the housing is provided, when viewed from a side, with a substantially regular polygon shape such as a triangle, a rectangle, a pentagon, a hexagon, a heptagon, or an octagon. In a further alternatively preferred embodiment the housing is provided, when viewed from a side, with a substantially regular polygon shape such as a star, a quadrilateral shape, or parallelogram shape.

The housing may be made from any suitable rigid, resilient or substantially resilient material; a plastics material is particularly preferred.

The membranes are attached to the housing, preferably to the front face thereof, such that one side of each membrane is exposed to the inner chamber of the housing and the other side of the membrane is exposed to the exterior of the housing.

Alternatively, where the dispenser has at least two membranes, said membranes may be disposed on the same or different walls or side(s) thereof, providing that, in use, the dispenser may be orientated such that at least one of said membranes is not in contact with the volatile material contained within the inner chamber.

The membranes may be attached such that they each form a leak-proof seal with the housing to prevent leaking of any volatile material through the seal when the dispenser is in use. The leak-proof seals may be particularly useful in preventing or minimising leaking of the volatile material when the dispenser has been knocked over or placed with the membrane(s) facing downwardly toward a surface on which the dispenser has been placed.

A preferred operating position is achieved where the dispenser is configured such that, in use, the membranes are not facing toward the surface on which the dispenser is placed. Even more preferably the dispenser is configured such that, in use, the membranes are facing in a substantially perpendicular direction to the surface on which the dispenser is placed.

The dispenser may be configured such that, in use, the preferred operating position is for the side edge or edges of the housing to contact the surface on which the dispenser is placed.

The housing may have one or more securing means protruding from or mounted on the exterior thereof. Said securing means may be adapted to engage the surface on which the dispenser is placed in order to secure it, in use, in one or more desired orientations with respect to the membranes and volatile material. Preferably the housing is provided with a plurality of securing means to permit the dispenser to be positioned a variety of orientations.

A barrier section is preferably located between adjacent membranes to prevent against uptake of volatile material, in use, by one membrane from an adjacent membrane or membranes. The barrier section may be provided by the structure or the housing and/or by suitable treatment of the membranes to prevent lateral uptake of the volatile material in use.

In preferred embodiments of the invention, the volatile material is present in a quantity in the dispenser that, when in use and the dispenser is orientated in a preferred operating position, the volatile material does not come into direct contact with the entirety of a discrete membrane. The precise provision of a specific quantity of volatile material in order to not contact the entirety of a discrete membrane is considered to be advantageous with respect to the ability of overcoming the build-up phenomena associated with the prior art.

The membranes are preferably constructed from a material that is vapour-permeable. Alternatively the membranes may be constructed from a material that is non-vapour-permeable. The selection of the particular properties of the membranes may alter depending on the particular volatile material the dispenser intends to emanate when in use. For instance, where the volatile material is a blend of components having greatly differing volatilities, it may be preferable to use a non-vapour-permeable membrane to provide a more consistent evaporation profile of the material.

The membranes are preferably made from one or more polymeric materials or polymeric materials in combination with inorganic materials, for example silica. Suitable polymeric materials may include but are not limited to polyethylene, polypropylene, polystyrene and or copolymers of the aforesaid and/or mixtures of any or all of the aforesaid materials.

The polymeric material used in the membranes of the present invention may further incorporate suitable components such as: fillers; plasticizers; antioxidants; lubricants; anti-statics; pigments; dyestuffs; stabilizers; light stabilizers; non-polymeric components and the like as well as mixtures thereof.

The membranes of the present invention may be provided with a thickness of between 0.01-10 mm. Preferably, the thickness is between 0.1-5 mm, and most preferably between 0.5-1.5 mm.

The dispenser of the present invention may have membranes of differing properties, by virtue of their chemical make-up and/or thickness, in order to provide the user with a greater variation to alter the passive or boosted emanation rate of the volatile material when the dispenser is in use, as described in more detail hereinafter.

The dispenser may be adapted to be operable, in use, to emanate a wide range of volatile materials, in particular volatile materials such as a fragrance/air freshener, an insecticide, a disinfectant, a bactericide, a fungicide and/or a medicament. In a preferred embodiment the dispenser is adapted such that, in use, it is operable to emanate a fragrance/air freshener.

The volatile material is preferably provided in liquid form.

Preferred liquid fragrance/air fresheners may include a pigment(s) and/or a colourant(s) in order to provide a visual indication to the user of the emanation occurring from the dispenser.

Suitable fragrance/air freshening volatile materials may comprises one or more fragrant components such as cedarwood, oil, sandalwood oil, bergamot, Bulgarian rose oil, patchouli, myrrh, clove leaf oil, linalol, ethyl alcohol, terpineol, menthol, citronellal, and/or phenyl ethyl alcohol.

Further fragrance/air freshening volatile materials that may offer suitable deodorant characteristic include one or more aroma and/or non-aroma chemicals which are known to have an action in reducing the perception of the intensity of malodours. In particular, such suitable materials may include: unsaturated esters, ketones, aldehydes, and/or a fragrant material e.g. citronellal and/or cedarwood oil (which is known to counteract the perception of tobacco malodour).

Further preferred liquid fragrance/air freshening volatile materials may comprise a malodour counteractant and/or an insecticide. Preferably it is the first fragrance composition which may further comprise a malodour counteractant and/or an insecticide.

A suitable insecticide for use in the present invention comprises one or more natural insecticides such as a pyrethroid, nicotinoid, rotenoid and/or one or more synthetic insecticides e.g. Metofluthrin®, Transfluthrin®, Tetramethrin®, Bioallethrin®, Allethrin®, phenthrin, a dinitrophenol, an organothiocyanate, benzene hexachloride, a polychlorinated cyclic hydrocarbon (e.g. Heptachlor®, Aldrin® and/or Telodrin®), and/or an organophosphorous (e.g. tetraethyl pyrophosphate).

The fragrance/air freshening volatile materials utilized in the present invention may further comprise an antioxidant such as tocopherol, ascorbyl palmitate, butylated toluene, ascorbic acid, teTt-butyl hydroquinone, beta carotene and/or a gallate. In these volatile materials may optionally comprise a UV stabiliser, such as Uvinol 400.

Turning to the mode of operation of the dispenser. According to the present invention, the dispenser is advantageously operable to passively emanate, in use, a volatile material therefrom, thus without the need of a propellant-powered aerosol or powered automation such as an electric heater, an electric fan or the like. Thus the dispenser of the present invention is capable of emanating, in use, merely by capillary/diffusion action of the volatile material across the membranes and evaporation into the external environment.

In use the dispenser may be orientated such that its side edge(s) are in contact with a surface on which the dispenser is placed. This orientation may permit the membranes to face in a substantially perpendicular direction to the surface. Indeed, this orientation may permit volatile material to be in direct contact with at least one discrete membrane whilst at least one membrane is not in direct contact with said material. The volatile material may then, via capillary/diffusion action, be transported across the membrane(s) which it is in contact with to permit emanation of the material by evaporation from the external surface of the membrane(s).

Whilst the above-described process will permit a continual emanation of the material, the dispenser may, advantageously, have the ability to boost the rate of emanation. The boosting of the emanation rate may be facilitated by a user lifting the dispenser, rotating it and placing back on to the surface such that a substantially opposite side edge(s) of the dispenser is in contact with the surface to the edge(s) that was in contact prior to moving the dispenser. This movement of the dispenser may cause the volatile material to move to a substantially opposite end of the inner chamber of the housing and, in doing so, come into direct contact with at least one membrane that was not in contact with the material prior to this movement. The result of the movement is that for a temporary period of time at least, at least two and possibly all of the membranes will be wetted with volatile material even though at least one membrane is not in direct contact therewith, temporarily permitting more evaporation of the volatile from the exterior surfaces of the membranes. For the membrane(s) that is no longer in direct contact with the volatile material, the quantity of material in these membrane(s) will eventually evaporate away therefrom and, thus, the boosting of the emanation rate will be over until the user moves the dispenser again.

It is considered that the boosting of the rate of emanation may, advantageously, overcome the aforementioned drawbacks of habituation, vacuum build-up and accumulation of volatile material components. Specifically, the movement of the volatile material within the chamber may alleviate any vacuum build-up due to the material moving away from the membrane or membranes suffering such a build-up, thus permitting more throughflow of air. The movement of volatile material may also impart a mixing effect on the components of the material such that the more volatile components are better dispersed throughout the material, thus leading, at least temporarily, to a more uniform emanation profile for all the components of the material.

The boosting of the emanation rate may increase the quantity of material emanating into the environment, thus potentially providing a user with noticeable boost of the material in the surrounding environment, particularly when the volatile material is a fragrance/air freshener, thus reducing the effect of habituation.

The presence of at least two discrete membranes may permit the user of the dispenser to better control the passive/boost emanation rate of the dispenser. In particular, the dispenser may be provided with more than two discrete membranes in order for the user to orientate the dispenser such that more or less of discrete membranes are in contact with the volatile material. Differently sized membranes may be provided to facilitate the user in selecting and controlling the passive/boost emanation rate. In such an arrangement a user may expose more discrete membrane(s) to the volatile material when a greater passive or boosted emanation rate is required.

The dispenser may be provided with an access port in the housing to permit the refilling of the level of volatile material. This feature, particularly where fragrances/air fresheners are concerned, may permit a user to alter the odour being emanated without the need to replace the entire dispenser.

The dispenser of the present invention may be configured to be operable for use with a refill of volatile material. The housing may be provided with a section thereof that is movable with respect to the other part or parts of the housing to permit a user access to the inner housing to position a refill therein. The housing may have a section which is hinged or connected by a snap-fit arrangement to allow it to be temporarily opened or removed to permit the loading of the refill.

The housing may be provided with additional sealing means between movable parts of the housing such that, after being opened and subsequently closed, the seal is sufficient to substantially completely prevent liquid and/or gaseous leakage of any volatile material from the inner chamber.

Preferably the side wall of the dispenser where the membranes are located is movable to permit the loading of a refill. This arrangement may be advantageous as a user will be better able to prevent spilling of the volatile material from the refill when loading this into the dispenser. In this arrangement a user would simply need to place the rear wall of the dispenser on a flat surface, remove or open the front wall of the dispenser with the membranes, load the refill into the inner chamber, remove any barrier material holding the volatile material in the refill, replace the front wall of the dispenser ensuring a secure seal with the rest of the housing and position the dispenser on one of its side edges to begin the passive emanation of the volatile material.

The inner surface of the movable part of the housing may be provided with one or more piercing means to further improve the anti-spill properties of the dispenser with a refill is loaded therein. In this arrangement the refill including any barrier material thereon would be loaded into the inner chamber and when the user replaced the movable part of the housing to securely seal it with the rest of the housing, the one or more piercing means would pierce the barrier material to permit the volatile material to contact the membrane(s) of the dispenser.

The refill is preferably shaped to substantially fill the inner chamber of the housing. The refill is preferably provided with a resilient housing having a open side wall covered with a removable or piercable barrier material.

According to a third aspect of the present invention therefore, there is provided method of manually boosting the emanation rate of a dispenser in accordance with the second aspect of the present invention, wherein a volatile material dispenser is manually moved from a first position to a second position such that the volatile material is in direct contact with at least one discrete membrane that said volatile material was not in contact with during said first position, and wherein at least one discrete membrane is not in direct contact with the volatile material in said second position.

### BRIEF DESCRIPTION OF THE DRAWINGS

Embodiments of the invention will now be described, by way of example only, with reference to the following drawings (FIGS. 1-5) in which:

Fig. A illustrates the evaporation rate of a volatile material versus time.

FIG. 1 illustrates a perspective view of a dispenser according to the present invention;

FIG. 2 illustrates the sequence of movement imparted to the dispenser of FIG. 1 when viewed from the side to activate the boost feature;

FIG. 3 illustrates a perspective view of a further dispenser according to the present invention;

FIG. 4 illustrates a perspective view of a yet further dispenser according to the present invention; and

FIG. 5 illustrates the varying orientation that may be imparted to the dispenser of FIG. 4 when viewed from the side to vary the passive emanation rate thereof.

### DESCRIPTION OF AN EMBODIMENT

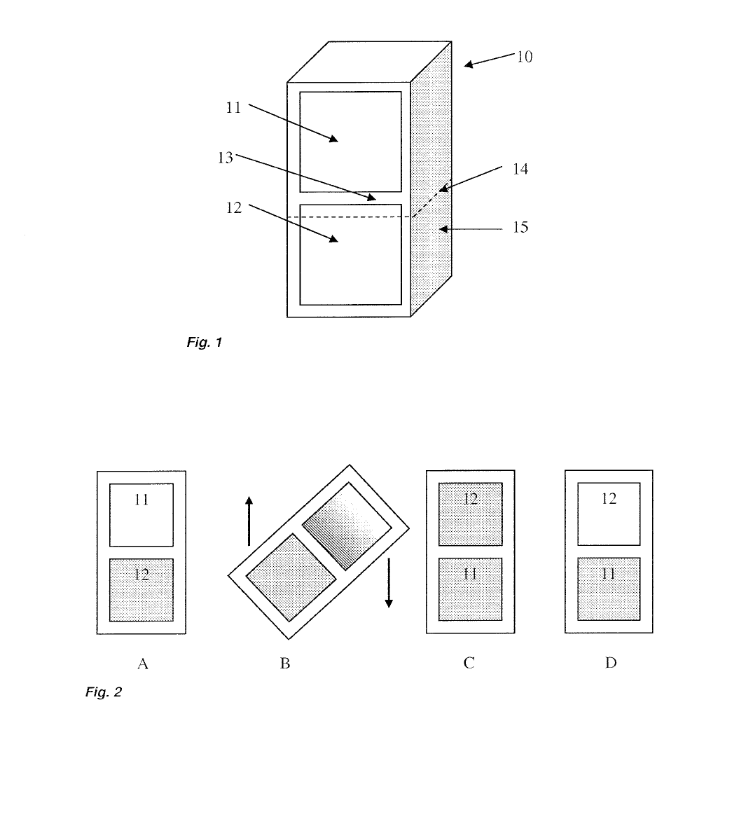

As can be seen in FIG. 1, a dispenser (10) according to an embodiment of the present invention is illustrated comprising a generally rectangular cube shape defined by a hollow housing with side edges (15) that space apart the front wall and rear wall (not shown) of the dispenser (10). In the front wall of the dispenser (10), are held two discrete membranes (11,12) separated by a separation member (13) of the housing. The membranes (11,12) are secured to the front wall of the housing to form a leak-proof seal therewith. A portion of the internal volume of the housing is filled with a volatile material, preferably a liquid volatile material. In the illustrated embodiment, the volatile material fills the internal volume of the housing up to the dashed line (14). This quantity of volatile material is preferred as it does not directly contact all of the membrane (12), thus likely permitting a general amelioration of vacuum effects as discussed hereinbefore.

Capillary/diffusion effects may cause the volatile material to be carried across the membrane (12) from its surface facing the interior of the housing to its surface facing the exterior of the housing. On reaching the exterior-facing surface of the membrane (12), the volatile material may be emanated into the surrounding environment by evaporation.

FIG. 1 illustrates a first position of the dispenser (10) and it is to be noted that one of the membranes (11) is not in direct contact with the volatile material.

The boost feature according to the present invention will now be described with reference to FIG. 2 where the movement of the dispenser (10) from a first position (position A) to a second position (position C,D) is demonstrated.

The movement of the dispenser (10) may be provided by a user lifting the dispenser (10), rotating it (position B) and placing back on to the surface such that a substantially opposite side edge(s) of the dispenser is in contact with the surface to the edge(s) that was in contact prior to moving the dispenser (position C,D). This movement of the dispenser may cause the volatile material to move to a substantially opposite end of the interior of the housing and, in doing so, move from direct contact with the membrane (12) and come into direct contact with the other membrane (11). The result of the movement is that for a temporary period of time (as shown in position C) both membranes (11,12) are wetted with volatile material even though only one membrane (11) is in direct contact therewith. With both membranes (11,12) wetted more evaporation of the volatile material from the exterior surfaces of both membranes (11,12) is permitted. For the membrane (12) that is no longer in direct contact with the volatile material, the quantity of volatile material in this membrane(12) will eventually evaporate away therefrom and, thus, once substantially completely evaporated away, the boost period will be over until the user moves the dispenser (10) again.

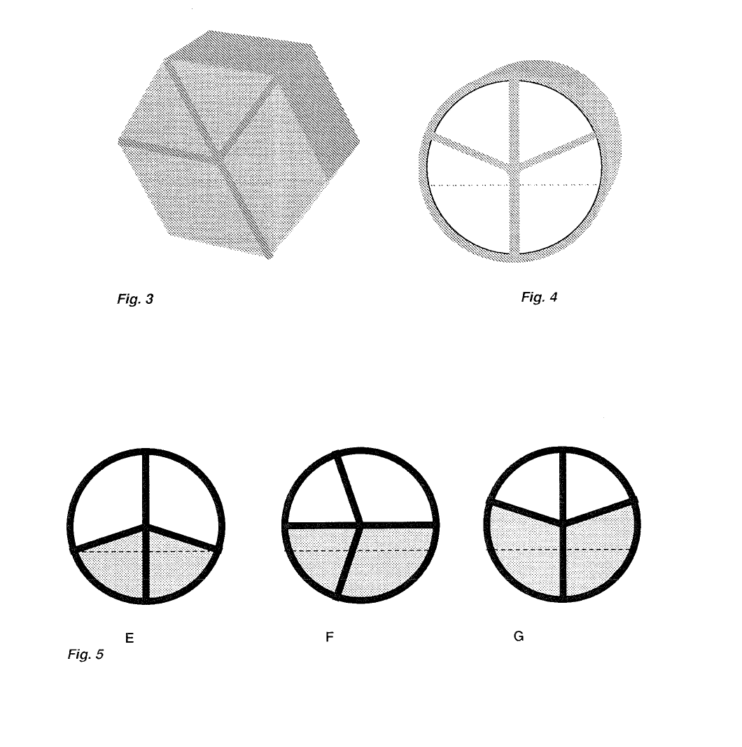

Although the dispenser (10) illustrated in FIGS. 1 & 2 is of a general cubical shape, FIGS. 3 & 4 illustrate that numerous shapes of housings and membranes have been envisaged within the various embodiments that are in accordance with the present invention.

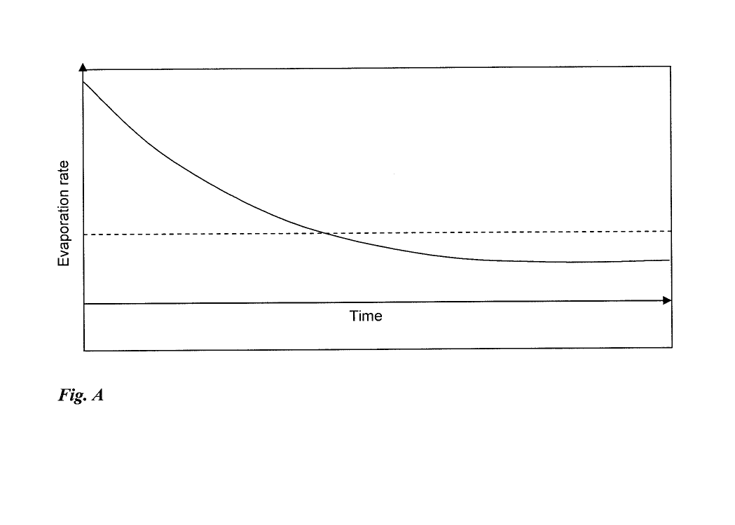

In FIG. 5 the particular orientation of a dispenser in accordance with the present invention illustrates how a user of the dispenser may alter the passive emanation rate of said dispenser.

In the illustrated embodiment, the dispenser is provided with numerous discrete membranes, in this case four membranes with two sets of sizes. If desired however, all of the discrete membranes (or portions of a membranes) could be of different sizes or a combination of sizes. The dashed line illustrates the level of volatile material held within the housing. As can be seen, a user may orientate the housing by altering which part of its side edge is in contact with the surface on which the housing stands. In so altering the orientation, different membranes are placed in direct contact with the volatile material. Where the membranes are of a different size, as is the case in FIG. 5, where the biggest membranes are in direct contact (see G), the greater the passive emanation rate of the volatile material will be. In contrast, where the smaller membranes are in direct contact with the volatile material (see E), the lower the passive emanation rate.

Thus, the embodiment illustrates one example of how a dispenser in accordance with the present invention may be operated to vary the emanation rate either passively by selecting the orientation of the dispenser, or actively by activating the boost feature.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. A passive dispenser for volatile material comprising a housing with an inner chamber configured to hold a quantity of volatile material therein, and further comprising more than two discrete membranes wherein said chamber is configured to permit direct contact between the volatile material and said membranes and wherein, in use, the user controls an emanation rate of the volatile material by orientating the dispenser such that more or less of the discrete membranes are in contact with the volatile material and such that at least one of said membranes is not in direct contact with the volatile material.

19. A dispenser according to claim 18, wherein volatile material is provided in the inner chamber.

20. A dispenser according to claim 18, wherein the housing of the dispenser comprises a front wall and a rear wall spaced apart by one or more side edges to define the inner chamber, and wherein the dispenser is configured such that, in use, an operating position is when the side edge or edges of the housing contact the surface on which the dispenser is placed.

21. A dispenser according to claim 20, wherein the membranes are attached to the front wall thereof, such that one side of each membrane is exposed to the inner chamber of the housing and the other side of the membrane is exposed to the exterior of the housing.

22. A dispenser according to claim 18, wherein the dispenser is configured such that, in use, the membranes are not facing toward the surface on which the dispenser is placed.

23. A dispenser according to claim 18, wherein the housing has one or more securing means protruding from or mounted on the exterior thereof, wherein said securing means are adapted to engage the surface on which the dispenser is placed.

24. A dispenser according to claim 18, wherein a barrier section is located between adjacent membranes.

25. A dispenser according to claim 20, wherein the volatile material is present in a quantity in the dispenser that, when in use and the dispenser is orientated in a preferred operating position, the volatile material does not come into direct contact with the entirety of a discrete membrane.

26. A dispenser according to claim 18, wherein the membranes have differing properties by virtue of their chemical make-up or thickness to provide the user, in use, with a greater variation to control the emanation rate.

27. A dispenser according to claim 18, wherein membranes of differently sized areas are provided to facilitate the user in selecting and controlling the emanation rate when using the dispenser.

28. A dispenser according to claim 18, wherein the dispenser is configured to be operable for use with a refill of volatile material.

29. A dispenser according to claim 18, wherein the housing is provided with a section thereof that is movable with respect to the other part or parts of the housing to permit a user access to the inner housing to position a refill therein, wherein the side wall of the dispenser where the membranes are located is movable to permit the loading of a refill.

30. A dispenser according to claim 29, wherein the inner surface of the movable part of the housing is provided with one or more piercing means

31. A dispenser according to claim 28, wherein the refill is shaped to substantially fill the inner chamber of the housing.

32. A method of manually boosting the emanation rate of a dispenser in accordance with claim 18, wherein a volatile material dispenser is manually moved from a first position to a second position such that the volatile material is in direct contact with at least one discrete membrane that said volatile material was not in contact with during said first position, and wherein at least one discrete membrane is not in direct contact with the volatile material in said second position.
